(19) United States
(12) Patent Application Publication
(10) Pub. No.: US 2003/0224485 A1
(43) Pub. Date: Dec. 4, 2003

(54) Expression system for ABC transporters

(76) Inventors: Robert S. Molday, Vancouver (CA); Jinhi Ahn, Vancouver (CA); William S. Hauswirth, Gainesville, FL (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/431,323

(22) Filed: May 6, 2003

Related U.S. Application Data
(60) Provisional application No. 60/391,644, filed on Jun. 27, 2002.

(30) Foreign Application Priority Data
May 6, 2002 (CA) 2,385,110

(51) Int. Cl. C07H 21/04; C12P 21/02; C12N 5/06; C07K 14/705

(52) U.S. Cl. 435/69.1; 435/320.1; 435/325; 530/350; 536/23.5

(57) ABSTRACT

The present invention provides a system and method for expressing a functional ABC (ATP-binding cassette) transporter in a host cell. A system comprises two or more expression vectors each comprising a nucleic acid molecule encoding one or more domains of the ABCR transporter gene and a means for expressing the nucleic acid molecule. Each expression vector of the system includes a nucleic acid molecule that encodes a domain that is functionally complementary to domains contained in the other expression vectors of the system but when taken together comprise the full ABCR transporter gene. Co-transfection of the expression vectors into a host cell provides co-expression of each of the domains of the protein which assemble to form an ABC transporter protein having functional characteristics of the full-length protein.

### FIELD OF THE INVENTION

The present invention relates to the field of protein expression. In particular, the present invention relates to the expression of large functional proteins.

### BACKGROUND

ABC (ATP-binding cassette) transporters represent the largest family of multi-spanning membrane proteins. These proteins bind ATP and use the energy to drive the transport of specific substrates across cell membranes. The chemical nature of the substrates handled by ABC transporters is extremely diverse, including drugs, lipids, peptides, metabolites and ions, yet ABC transporters are highly conserved.

Proteins are classified as ABC transporters based on the sequence and organization of their nucleotide-binding domain(s) (NBDs), which are responsible for binding and hydrolyzing ATP. The NBDs are highly conserved and contain characteristic motifs; Walker A and B, found in all ATP-binding proteins, as well as the C motif unique to the ABC transporters. Typically, ABC transporters consist of four “core” domains, two multi-spanning membrane domains (MSDs) that serve as a pathway for the translocation of a substrate across membranes and two ATP-binding cassettes or nucleotide binding domains (NBDs) that provide the energy for substrate transport (Higgins, C. F. (1992) Annu Rev Cell Biol 8, 67-113).

In eukaryotic ABC transporters, these domains are typically found either on a single long polypeptide chain (full transporters) as in the case of CFTR and the multi-drug resistance proteins, P-glycoprotein and MRP 1, or as a complex of two identical or similar ‘half molecule’ subunits each having a MSD and a NBD (half transporters), as found in the TAP1/TAP2 ABC transporter associated with peptide antigen processing. ABCR belongs in the first category since it consists of a single 2273 amino acid polypeptide comprised of two tandemly arranged halves (Illing, M., et al. (1997) J Biol Chem 272(15), 10303-10; Allikmets, R. et al. (1997) Nature Genet. 15, 236-246). Each half contains a MSD followed by a cytoplasmic NBD. A distinguishing feature of ABCR and other members of the ABCA subfamily is the presence of a large exocytoplasmic (extracellular/lumen) domain that connects the first transmembrane segment to the multi-spanning membrane domain in each half of the protein (Illing, M., et al. (1997) J Biol Chem 272(15), 10303-10; Bungert, S., et al. (2001) J Biol Chem 276(26), 23539-46; Fitzgerald, M. L., et al. (2002) J Biol Chem 277(36), 33178-87).

There are 48 or so mammalian ABC transporters that are known, and these have been divided into subfamilies based on similarity of gene structure (full or half transporters), order of the domains, and on sequence homology in the NBDs and MSDs. The mammalian ABC transporters have been divided into seven subfamilies: ABCA, ABCB, ABCC, ABCD, ABCE, ABCF, and ABCG, each comprising members exhibiting a particular function (Dean, M. Rzhetsky, A., Allikments, R. (2001) Genome Research 11:1156-1166). Mutations in the genes encoding many of these 48 or so ABC transporters are associated with a variety of inherited diseases such as cystic fibrosis, adrenoleukodystrophy, Tangier disease, and obstetric cholestasis. As well, overexpression of certain ABC transporters is the most frequent cause of resistance to cytotoxic agents including antibiotics, antifungals, herbicides, and anticancer drugs (Higgins et al. (2001) Science. 293:1782-1784).

ABCR (ABCA4), formerly known as the rim protein, is a member of the ABCA subclass of ATP-binding cassette (ABC) transporters that is principally found along the rims and incisures of rod and cone photoreceptor outer segment disk membranes (Papermaster, D. S., et al. (1982) Vision Res 22(12), 1417-28; Illing, M., et al. (1997) J Biol Chem 272(15), 10303-10; Molday, L. L., et al. (2000) Nat Genet 25(3), 257-8; Sun, H., et al. (1997) Nature Genet. 17, 15-16). It is thought to function in the transport of all-trans retinal or flipping of all-trans retinylidene-phosphidylethanolamine across the disk membrane following the photobleaching of rhodopsin (Sun, H., et al. (1999) J Biol Chem 274(12), 8269-81; Ahn, J., et al. (2000) J Biol Chem 275(27), 20399-405; Weng, J., et al. (1999) Cell 98, 13-23). This transport process facilitates the reduction of all-trans retinal to all-trans retinol by retinol dehydrogenase on the surface of disk membranes as a key step in the recycling of all-trans retinal to 11-cis retinal for regeneration of rhodopsin and cone opsin.

The importance of ABCR in photoreceptor biology is highlighted by the finding that over 300 mutations in the ABCR gene have been associated with a variety of clinically distinct autosomal recessive inherited retinal degenerative diseases including Stargardt macular dystrophy, fundus flavimaculatus, cone-rod dystrophy, and retinitis pigmentosa (Allikmets, R., et al. (1997) Nature Genet. 15, 236-246; Nasonkin, I., et al. (1998) Hum. Genet. 102, 21-26; Martinez-Mir, A., et al. (1998) Nature Genet. 18, 11-12; Cremers, F. P., et al. (1998) Hum Mol Genet 7(3), 355-62; Lewis, R. A., et al. (1999) Am J Hum Genet 64(2), 422-34; Rivera, A., et al. (2000) Am J Hum Genet 67(4), 800-13; Souied, E. H., et al. (1999) Invest. Ophthalmol. Vis. Sci. 40(11), 2740-2744; Allikmets, R. (2000) Am J Hum Genet 67(4), 793-9.). Stargardt macular dystrophy, the most common disease associated with mutations in ABCR, is an early onset disease characterized by progressive loss of central vision, delayed dark adaptation, accumulation of yellow deposits known as lipofuscin within the central retina, and atrophy of macular region of the retina and underlying retinal pigment epithelial cells. Abcr knockout mice exhibit many of these features including delayed dark adaptation, accumulation of lipofuscin deposits containing the diretinyl compound A2E in retinal pigment epithelial cells, and slow progressive photoreceptor degeneration (Weng, J., et al. (1999) Cell 98, 13-23).

Genes encoding most mammalian ABC transporters are very large in size coding for transporters that are typically between 120 kDa to 250 kDa in size. The human ABCR gene, for example, is over 6.8 kb in size and codes for a protein of 2,272 amino acids which is expressed specifically in rod and cone photoreceptor cells of the human retina (Molday, L. L., et al. (2000) Nat Genet 25(3), 257-8). Due to their large size, most ABC transporter genes cannot be packaged into standard expression vectors for transgenic expression of this family of proteins.

Most expression vector systems are limited in the size of genetic material which may be inserted. For example, recombinant adeno-associated viral (rAAV) vectors, which are useful vectors for gene therapy applications, have an insert capacity of 4.9 kb, which must include not only the gene, but the necessary promoters and regulatory elements as well. This limits the types of genes that may be effectively packaged into expression vectors for successful transfection of host cells. As a result, there is a need for transgenic expression systems capable of mediating the transfer and expression of large proteins such as the ABC transporters.

One example that circumvents the problem of delivering transgenes that exceed the normal packaging size of the expression vector, is provided by WO 01/25465 A1. The method comprises splitting either components of the transcription regulatory unit or the transgene itself and packaging these parts in two recombinant adeno-associated viral (rAAV) vectors. Co-infection with both rAAV vectors is described to result in the reconstruction of intact expression cassettes through inverted terminal repeat mediated intermolecular concatamerization. This method is limited, however, to expanding the packaging capacity of the viral vector system at the nucleotide level.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a system and method for expressing an ABC transporter in a host cell.

In accordance with one embodiment of the present invention, there is provided a nucleic acid composition for expression of a functional ABCR transporter in a host cell, said nucleic acid composition comprising two or more different nucleic acid molecules, each nucleic acid molecule encoding one or more domains of an ABCR transporter, wherein said at least one of the domains encoded by each nucleic acid molecule are functionally complementary.

In accordance with another embodiment of the present invention, there is provided a method of expressing a functional ABCR transporter in a host cell comprising transforming or transfecting said host cell with the nucleic acid composition of the instant invention.

In accordance with another embodiment of the present invention, there is provided a system for expressing an ABCR transporter in a host cell comprising two or more expression vectors, each expression vector comprising a different nucleic acid molecule and each nucleic acid molecule encoding one or more domains of an ABCR transporter, wherein said at least one of the domains encoded by each nucleic acid molecule is a functionally complementary domain, and wherein, upon co-expression in said host cell, the functionally complementary domains associate to provide a functional ABCR transporter.

In accordance with a further embodiment of the present invention, there is provided a host cell comprising the nucleic acid composition or system of the instant invention.

In accordance with another embodiment of the present invention, there is provided a method for expressing an ABCR transporter in a host cell comprising:

(a) transforming or transfecting said host cell with two or more expression vectors, each expression vector comprising a different nucleic acid molecule and each nucleic acid molecule encoding one or more domains of an ABCR transporter; and

(b) culturing said host cell under conditions that allow for expression of said one or more domains.

In accordance with a further embodiment of the present invention, there is provided a kit for expressing an ABCR transporter in a host cell comprising:

(a) the nucleic acid composition or the system of the instant invention;

(b) one or more containers, and optionally

(c) instructions for use.

### BRIEF DESCRIPTION OF THE FIGURES

FIG. 1. Immunofluorescence localization of ABCR in COS-1 cells. Cells transfected with constructs coding for the full-length ABCR, N Half (amino acids 1-1325), C Half (amino acids 1326-2273), both halves (NC Halves) were labeled with Rim5B4 (which binds an 8 amino acid epitope in NBD1 of the human ABCR; N-half) or Rim3F4 (which binds 9 amino acids near the C-terminus of ABCR) and Cy3 conjugated anti-mouse immunogloubin for analysis by immunofluorescence microscopy. Full-length ABCR and co-expressed NC halves localize to both intracellular vesicles and the endoplasmic reticulum (ER)-Golgi network. The N and C halves localize predominantly in the ER-Golgi network.

FIG. 2. The N- and C-terminal halves of ABCR associate when co-expressed in COS-1 cells. Cells transfected with the full-length ABCR, C-half, N-half, or co-transfected with the N and C halves (NC) were harvested and the proteins were solubilized and immunopurified on a Rim3F4-Sepharose matrix. ABCR and half molecules were eluted from the matrix with 3F4 peptide and analyzed by SDS-PAGE and Western blotting. The upper blot was probed for the Rim5B4 (reactive to the N-half) and the lower blot was probed with both Rim5B4 and Rim3F4 (reactive for the C-half). Lanes 1, 4, 7, 10 13: solubilized COS-1 cell lysate; lanes 2, 5, 8, 11, 14: flow-through fraction from Rim 3F4 column; lanes 3, 6, 9, 12, 15: peptide eluate. CB: Commassie blue stained gel showing purified full-length ABCR and co-expressed halves. The positions of the full-length ABCR (220 kDa), N-half (140 kDa) and C-half (10 kDa) are indicated by arrows. In lanes 13-15 (N+C), cells expressing only N-half were mixed with cells expressing only C-half after detergent solublization. Under these conditions, the N-half did not co-purify with the C-half.

FIG. 3. Both halves of ABCR are required for retinal-stimulated ATPase activity. Purified ABCR or half molecules were expressed in COS-1 cells, purified on Rim3F4-Sepharose or Rho1D4-Sepharose (for 1D4-tagged N half), and reconstituted in liposomes. The ATPase activity was measured as a function of all-trans retinal. Filled circle, full-length ABCR; open circle, co-expressed and co-purified N and C halves; open triangle, individually expressed and purified C-half; filled triangle, individually expressed and purified 1 D4-tagged N-half. The data are averages from at least three experiments.

FIG. 4. Lysine to methionine Walker A mutations in ABCR abolish ATP hydrolysis but not ATP binding. A. ATPase activity was measured in the absence (white bars) or presence of 50 μM all-trans retinal (grey bars). WT, wild-type; K969M in NBD1; K1978M in NBD2; MM, K969M/K1978M double mutant; NCM, N-half co-expressed with C-half containing a K1978M mutation. B. ATP photoaffinity labeling was carried out by irradiating membranes from COS-1 cells expressing wild-type (WT), K969M, K1978M, or K969M/K1978M double mutant (MM) with 3 μM 8-azido-[α-³²P]ATP. The labeled protein was isolated with Rim3F4-Sepharose and separated on SDS gels. Left panel: gel stained with Coomassie blue. Right panel: corresponding ³²P labeling obtained with a phosphorimager. All mutant proteins were labeled with 8-azido-[α-³²P]ATP, but the double mutant bound less nucleotide.

FIG. 5. Azido-ATP photoaffinity labeling of the N and C halves of ABCR. A. Membranes from transfected cells expressing either full-length ABCR (lanes 1 & 2) or co-expressing the N and C halves (lanes 3 & 4) were photoaffinity labeled with 1.5 μM 8-azido-[α-³²P]ATP in the absence or presence of 1 mM ATP. The expressed protein was isolated on a Rim3F4-Sepharose matrix prior to analysis by SDS-PAGE and phosphorimage analysis. Left panel: Coomassie blue stained gel. Right panel: Azido-ATP labeling. B. Membranes from transfected cells expressing full-length ABCR, the N-half, the C-half, or both halves (NC halves) were labeled with 8-azido-[α-³²P] and isolated as above. Similar amounts of protein were loaded in each lane of the gel as judged by staining with Coomassie brilliant blue (not shown). C. Rod outer segment membranes were incubated with (+) or without (−) trypsin and subsequently labeled with azido-[α-³²P]ATP. ABCR and the associated N and C complex were purified on a Rim3F4-Sepharose matrix. Left panel: Azido-ATP labeling of the full-length ABCR (220 kDa) and the C-half (14 kDa); N-half was not labeled. Right panel: Western blots labeled for the full-length ABCR and C-half with the Rim3F4 antibody and the N-half with the Rim5B4 antibody.

FIG. 6. Azido-ATP binding to the N and C-halves of ABCA1. Membranes from cells expressing the N and C halves of ABCA1 engineered to contain the 3F4 epitope in the C-half and ABCR were photoaffinity labeled with azido-[α−³²P]ATP, isolated by immunoprecipitation and analyzed on an SDS gel. Coomassie blue stained gel (left panel) and azido-ATP labeling of co-expressed N and C halves of ABCA1 (lane 1) and ABCR (lane 2) isolated on a Rim3F4-Sepharose matrix, and ABCR (lane 3) isolated on a Rim5B4-Sepharose matrix. Molecular weight markers are shown on the left. The positions of the N and C halves are indicated by arrows on the right. Both the N and C halves of ABCA1 label with 8-azido-ATP, where as only the C-half of ABCR is intensely labeled.

FIG. 7. Nucleotide trapping by ABCR and co-expressed N and C halves. A. Effect of orthovanadate on nucleotide trapping. Membranes from transfected cells were incubated with 5 μM 8-azido-[α-³²P]ATP at 37° C., washed, and tightly bound nucleotides were UV crosslinked. Lane 1, full-length ABCR; lane 2, full-length ABCR incubated with 800 μM orthovanadate; lane 3, co-expressed N and C halves; lane 4, co-expressed N- and C-halves incubated with 800 μM orthovanadate. B. Effect of temperature and retinal on nucleotide trapping. Lane 1, full-length ABCR labeled at 0° C.; lane 2, full-length ABCR labeled at 37° C.; lane 3, full-length ABCR labeled at 37° C. in the presence of 50 μM all-trans retinal; lane 4, co-expressed N and C halves labeled at 0° C.; lane 5, co-expressed N and C halves labeled at 37° C.; lane 6, co-expressed N and C halves labeled at 37° C. in the presence of 50 μM all-trans retinal.

### DETAILED DESCRIPTION OF THE INVENTION

ABC Transporters

The present invention provides a system and method for expressing an ABC transporter in a host cell. In accordance with the present invention, two or more functionally complementary domains from an ABC transporter are co-expressed in a host cell and associate in the host cell to form a functional ABC transporter. As used interchangeably herein, the terms “functionally complementary domain” and “complementary domain” refer to a discrete part of a polypeptide, i.e. a domain, that functionally interacts, for example by non-covalent association, with a second, different domain to produce a fully functional protein. The second domain may be part of the same polypeptide or it may be part of a separate polypeptide. In one embodiment, the functionally complementary domains are from an ABCR transporter, a member of the ABCA subfamily. In another embodiment, the functionally complementary domains comprise a nucleotide binding domain (NBD) of an ABCR transporter. In a further embodiment, the functionally complementary domains comprise a multi-spanning membrane domain (MSD) and a nucleotide binding domain (NBD) of an ABCR transporter.

Nucleic Acid Molecules

In accordance with the present invention, nucleic acid molecules encoding at least one functionally complementary domain of an ABC transporter are isolated. By “isolated”, it is meant a nucleic acid molecule of genomic, cDNA, RNA, or synthetic origin or some combination thereof, which is no longer associated with the cell in which the nucleic acid molecule is found in nature. The nucleic acid molecules of this invention may be isolated from cDNA or genomic libraries or directly from isolated eukaryotic DNA using standard techniques [see, for example, Ausubel et al., Current Protocols in Molecular Biology, Wiley & Sons, NY (1997 and updates); Sambrook et al., Molecular Cloning: A Laboratory Manual, Cold-Spring Harbor Press, NY (2001)].

Nucleic acid molecules of this invention further include sequences having substantial sequence similarity to a nucleic acid encoding a functionally complementary domain of an ABC transporter. The term “substantial similarity” or “substantial sequence similarity,” when referring to a nucleic acid or fragment thereof, indicates that, when optimally aligned (with appropriate nucleotide insertions or deletions) with another nucleic acid (or its complementary strand), there is nucleotide sequence identity in at least about 50% of the nucleotide bases. In one embodiment of the invention, substantial sequence similarity refers to nucleotide sequence identity in at least about 60% of the nucleotide bases. In another embodiment, in at least about 70% of the nucleotide bases. In other embodiments, in at least about 80%, at least about 90%, and at least about 95-98% of the nucleotide bases, as measured by any well-known algorithm of sequence identity, such as FASTA, BLAST or Gap.

Nucleic acid sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998)). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1.

Alternatively, substantial similarity exists when a nucleic acid or fragment thereof hybridizes to another nucleic acid, to a strand of another nucleic acid, or to the complementary strand thereof, under selective hybridization conditions. Typically, selective hybridization will occur when there is at least about 55% sequence identity. In one embodiment of the invention, selective hybridization occurs when there is at least about 65% sequence identity. In other embodiments, there is at least about 75%, and at least about 90% sequence identity. Sequence identity is measured over a stretch of at least about 14 nucleotides. In one embodiment sequence identity is measured over at least 17 nucleotides. In other embodiments, over at least 20, 25, 30, 35, 40, 50, 60, 70, 80, 90 and 100 nucleotides.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. As a general guideline, stringent washing conditions tend to fall within the ranges: 1-3×SSC, 0.1-1% SDS, 50-70° C. with a change of wash solution after about 5-30 minutes.

As defined herein, nucleic acid molecules that do not hybridize to each other under stringent conditions are still substantially similar to one another if they encode polypeptides that are substantially identical to each other. This occurs, for example, when a nucleic acid molecule is created synthetically or recombinantly using high codon degeneracy as permitted by the redundancy of the genetic code.

It will be recognized by one of ordinary skill in the art that nucleic acids of this invention may be modified using standard techniques of site specific mutagenesis or PCR, or modification of the sequence may be accomplished in producing a synthetic nucleic acid sequence. Such modified sequences are also considered in this invention. For example, due to the degeneracy of the genetic code, which is well-known to the art (i.e., for many amino acids, there is more than one nucleotide triplet which serves as the codon for the amino acid) codons may be changed such that the nucleic acid sequence encodes the same amino acid sequence, or alternatively, codons may be altered such that conservative amino acid substitutions or substitutions of similar amino acids result without affecting protein function.

The present invention also contemplates genetic engineering of the nucleic acid molecules encoding a functionally complementary domain such that one or more of the encoded amino acids are substantially altered. Genetic engineering techniques are standard in the art. The insertion or substitution of amino acids can be accomplished without adversely affecting the function of the domain (for example, by altering amino acids at one or more positions remote from the functional region(s) of the protein), or the inserted or substituted amino acid(s) may enhance the function of the domain, for example, inserted or substituted amino acid(s) may enhance the ATP binding ability or ATPase activity of the associated protein, or they may enhance the association between two domains. Alternatively, the inserted or substituted amino acids may constitute a marker peptide or tag, such as an epitope.

In one embodiment of the present invention, the nucleic acid molecule encoding a functionally complementary domain is genetically engineered to include an epitope. In another embodiment, the nucleic acid molecule encoding a functionally complementary domain is genetically engineered to include a 3F4, 5B4 or 1D4 epitope.

The present invention also contemplates nucleic acid molecules encoding a functionally complementary domain fused to a heterologous nucleic acid encoding a heterologous polypeptide. Typically such heterologous nucleic acids are fused in frame to the 5′ or 3′ end of the nucleic acid encoding the functionally complementary domain and are thus capable of expressing a fusion protein comprising the functionally complementary domain and the heterologous polypeptide. It will be understood that such heterologous polypeptides will not interfere with the functioning of the functionally complementary domain. Examples of useful heterologous polypeptides that may be included in the fusion proteins of the present invention include those designed to facilitate purification and/or visualization of expressed functionally complementary domains.

Unless otherwise specified, the nucleic acid molecules of the present invention are prepared in such a manner that the intrinsic activity of the encoded domain is retained. The nucleic acid molecules encoding different functionally complementary domains of an ABC transporter can be used directly to transform an appropriate host cell or they may be first incorporated into an appropriate expression vector. Methods of transforming host cells with “naked” nucleic acid molecules are known in the art and include, but are not limited to, direct injection of the naked nucleic acid molecule (Felgner, P. L. and G. Rhodes, (1991) Nature 349:351-352; U.S. Pat. No. 5,679,647) or the nucleic acid molecule formulated in compositions with other agents which may facilitate its uptake by the cell, including saponins (U.S. Pat. No. 5,739,118) and cationic polyamines (U.S. Pat. No. 5,837,533); use of microparticle bombardment (for example, by use of a “gene gun”; Biolistic, Dupont); coating or complexing the nucleic acid with lipids, cell-surface receptors or transfecting agents and encapsulation in liposomes, microparticles or microcapsules.

The nucleic acid molecule can be operably linked to one or more regulatory elements that enhance expression of the encoded ABC domain. “Regulatory elements” or “regulatory sequences” refer to polynucleotide sequences that are necessary to effect the expression of coding and non-coding sequences to which they are linked, or that enhance transcription or translation of the sequences, stabilize the transcribed mRNA or otherwise contribute to the efficient expression of the encoded polypeptide. The nature of such regulatory elements differs depending upon the host organism; in prokaryotes, such control sequences generally include promoter, ribosomal binding site, and transcription termination sequence; in eukaryotes, generally, such regulatory elements include promoters and transcription termination sequence. Regulatory elements can further include enhancers, internal ribosomal entry sites and polyadenylation signals. Specific initiation signals may also be required for efficient translation of inserted nucleic acid sequences. As is known in the art, these signals include the ATG initiation codon and adjacent sequences. A minority of genes have a translation initiation codon having the sequence 5′-GTG, 5′-TTG or 5′-CTG, and 5′-ATA, 5′-ACG and 5′-CTG have been shown to function in vivo. These alternative initiation codons are also contemplated by the present invention.

One skilled in the art will appreciate that selection of suitable regulatory elements is dependent on the host cell chosen for expression of the nucleic acid and that such regulatory elements may be derived from a variety of sources, including bacterial, fungal, viral, mammalian or insect genes. The term “regulatory elements” is intended to include, at a minimum, components whose presence can influence expression of the inserted nucleic acid sequences, and can also include additional components whose presence is advantageous, for example, leader sequences and fusion partner sequences.

Persons of skill in the art will understand that a first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequences. Generally, operably linked DNA sequences are contiguous and, where necessary to join two protein coding regions, maintain the correct reading frame.

A promoter, as used herein, is a DNA sequence in a gene, usually (but not necessarily) upstream (5′) to its coding sequence, which controls the expression of the coding sequence by providing the recognition for RNA polymerase and other factors required for proper transcription. The type of promoter is dependent upon the vector and the host cell selected and can be readily determined by one skilled in the art. The promoter can be of prokaryotic and eukaryotic origin, or it may be the native promoter for the ABC transporter gene. In one embodiment of the present invention, the promoter is a eukaryotic promoter. Examples of suitable eukaryotic promoters include inducible eukaryotic promoters, e.g. tetO-minimal CMV, inducible human metallothionein IIa gene enhancer/promoter, and constitutive eukaryotic promoters e.g. CMV promoter, SV40 late promoter, RSV LTR (rous sarcoma virus long terminal repeat) promoter, and BGH (bovine growth hormone) promoter, although many other promoter elements well known in the art may be employed in the practice of the invention. The present invention also contemplates the use of the native promoter from the ABCR transporter gene.

Expression Vector

In accordance with one embodiment of the present invention, nucleic acid molecules each encoding at least one functionally complementary domain of an ABC transporter are each separately incorporated into an expression vector. Examples of suitable expression vectors include, but are not limited to, plasmids, phagemids, cosmids, bacteriophage, bacterial artificial chromosomes (BAC), yeast artificial chromosomes (YAC), baculoviruses, viral vectors (such as replication defective retroviruses, adenoviruses and adeno-associated viruses) or DNA viruses. In one embodiment of the present invention, the nucleic acid encoding the ABC transporter domain is cloned into a plasmid. In another embodiment, the nucleic acid is cloned into a viral vector.

In one embodiment of the present invention, each vector comprises a nucleic acid molecule encoding a functionally complementary domain of an ABCR transporter operably linked to one or more regulatory elements. “Regulatory elements” contemplated by the present invention for this purpose include those described above and may be associated with the nucleic acid prior to insertion into the vector or they may be associated with the vector.

Recombinant expression vectors can be constructed by standard techniques known to one of ordinary skill in the art and found, for example, in Sambrook et al. (1989) in Molecular Cloning: A Laboratory Manual. A variety of strategies are available for ligating molecules of DNA, the choice of which depends on the nature of the termini of the DNA molecules and can be readily determined by persons skilled in the art. The vectors of the present invention may also contain other heterologous nucleic acid sequences to facilitate vector propagation and selection in host cells. Coding sequences for selectable markers, and reporter genes are well known to persons skilled in the art.

Transformation or Transfection into a Host Cell

The recombinant expression vectors of the present invention are introduced into a host cell capable of expressing the protein coding region contained in each of the recombinant expression vectors. The precise host cell used is not critical to the instant invention and will depend upon the expression vector selected. Examples of suitable host cells include, but are not limited to, prokaryotic host cells (e.g., E. coli or B. subtilis) and eukaryotic host cells (e.g., Saccharomyces or Pichia; mammalian cells, e.g., COS, NIH 3T3, CHO, BHK, 293, or HeLa cells; insect cells or plant cells). In one embodiment of the present invention, the host cell is of mammalian origin.

The expression vectors can be introduced into a suitable host cell via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2nd Edition, Cold Spring Harbor Laboratory press (1989)), and other laboratory manuals.

The ABC transporter proteins of the present invention can optionally be purified from the host cells by standard techniques known in the art. To confirm the presence of the preselected DNA sequence in the host cell, a variety of assays may be performed. (see, for example, Ausubel et al., Current Protocols in Molecular Biology, Wiley & Sons, NY). Such assays include, for example, “molecular biological” assays well known to those of skill in the art, such as Southern and Northern blotting, RT-PCR and PCR; “biochemical” assays, such as detecting the presence of a polypeptide expressed from a gene present in the vector, e.g. by immunological means (immunoprecipitations, immunoaffinity columns, ELISAs and Western blots), by conducting activity assays or other assays useful to identify molecules falling within the scope of the invention.

Association of Functionally Complementary Domains to form an ABC Transporter

The co-expressed functionally complementary domains can be assayed to determine association of the domains to form a functional ABC transporter using standard techniques known in the art. Exemplary testing methods are outlined herein and are not intended to limit the scope of the present invention.

In accordance with the present invention, the functionally complementary domains are considered to have associated to form a functional ABC transporter if at least 20% of the total recombinant protein isolated from a cell is in the form of the assembled transporter protein. In one embodiment, at least 30% of the total recombinant protein from a cell is in the form of the assembled transporter protein. In other embodiments, at least 40% and at least 50% of the total recombinant protein from a cell is in the form of the assembled transporter protein.

Immunofluorescence Microscopy

Functional association of the domains of a co-expressed ABC transporter may be determined, for example, by indirect immunofluorescence microscopy. Like a full-length ABC transporter, co-expressed functionally complementary domains that have associated into an ABC transporter should exit from the ER to intracellular vesicles, indicating that the protein complex is properly folded and assembled so as to pass through the quality control system of the ER. In contrast, domains which fail to associate, or which are individually expressed, will be misfolded and are, as a result, retained in the ER.

Immunofluoreseence microscopy techniques for localizing proteins within a cell are well known in the art. Typically, cells are first treated with a primary antibody that recognises a specific epitope within one or more of the functionally complementary domains. The epitope may be a part of the natural sequence or it may have been genetically engineered into the domain as described above. The cells are then treated with a secondary antibody that specifically binds the primary antibody and that is conjugated to a fluorescent dye. Subsequent visualization of the dye by fluorescence microscopy allows for the localization of the expressed domain. Examples of useful dyes for fluorescence microscopy include, but are not limited to, rhodamine, Texas red, Cy3, Cy5 and fluorescein. The use of two or more primary antibodies specific to different epitopes, which are either naturally present or have been engineered into the separate co-expressed functionally complementary domains, together with secondary antibodies each conjugated to a fluorescent dye that fluoresces at a different wavelength permits the localization of multiple domains within a cell.

Membrane Insertion

The ability of the functionally complementary domains to associate and insert into a membrane can be analyzed in vitro. Typically the expressed protein is solubilized using detergents and then reconstituted into membrane vesicles using standard techniques such as those described in Molday et al. (J. Biol. Chem., (1999) 274:8269-2681); Ahn and Molday (Methods in Enzymology, (2000) 315:864-879) and the Examples.

Immunoaffinity Assays

Specific epitopes naturally present or genetically engineered into one or more of the functionally complementary domains can also be used to determine association of co-expressed domains into an ABC transporter by immunoaffinity assays. A monoclonal antibody directed against a defined epitope on one domain of a co-expressed functional ABC transporter can be coupled to a suitable matrix and contacted with, for example, cell extracts from cells co-expressing the functionally complementary domains. Subsequent washing of the matrix is followed by elution of the bound protein with a suitable releasing agent, such as a peptide form of the epitope. The released protein fraction can then be analyzed by standard techniques, such as SDS-PAGE, size exclusion chromatography, native PAGE, mass spectrometry and the like. The presence in the eluted fraction of both the domain exhibiting the targeted epitope and one or more co-expressed domains that do not exhibit the epitope is indicative of association of the co-expressed functionally complementary domains in the cell. A domain which has not associated to form the functional ABC transporter protein, and which does not exhibit the epitope, will form the “flow-through fraction,” which is removed in the wash step(s). Such affinity assays are known in the art, as are methods of coupling antibodies to a suitable matrix (see for example, Coligan et al., (eds.) Current Protocols in Protein Science, and Current Protocols in Immunology, J. Wiley & Sons, New York, N.Y.). Suitable matrices include, but are not limited to, various chromatographic resin beads (including, for example, Sepharose-, agarose- and cellulose-based resins), microtitre or cell culture plates, magnetic beads, and the like.

Cross-Linking Experiments

Methods of chemically cross-linking proteins are known in the art and include, for example, the use of cross-linking agents such as glutaraldehyde, disuccinimidyl suberate, ethylene glycol bis(succinimidylsuccinate), bis[2-(succinimidooxycarbonyloxy)ethyl]sulfone), dithiobis(succinimidylpropionate), M-maleimidobenzoyl succinimide ester and N-hydroxysuccinimide. A large number of other cross-linking agents are known and are commercially available (for example, from Pierce Biotechnology, Rockford, Ill.). Methods of cross-linking proteins that take advantage of the properties of enzymes and the presence of certain residues in the protein are also known. For example, zero-order cross-linking takes advantage of the activity of the enzyme transglutaminase to cross-link lysine and glutamine residues in the protein that are close together in three-dimensional space.

The ability of the functionally complementary domains to associate and form an ABC transporter can thus be determined by treating membranes isolated from cells co-expressing the domains with an appropriate cross-linking agent using standard techniques and then analyzing for the cross-linked protein by SDS-PAGE. The presence of a protein having a molecular weight corresponding to the molecular weight of the native ABC transporter is indicative of the association of the functionally complementary domains.

As cross-linking of the expressed ABC transporter protein may render the protein inactive, the activity of the protein may be assayed, using techniques such as those described below, prior to the cross-linking experiments if desired. For example, the expressed transporter could be first photoaffinity labelled with an ATP derivative (such as 8-azido-ATP) and then cross-linked. Subsequent SDS-PAGE and detection of the labelled ATP showing that the ATP is associated only with the high molecular weight associated transporter protein would demonstrate that the functionally complementary domains associate and form an active transporter.

Functional Protein Activity

The functional activity of the ABC transporter proteins may be evaluated by using standard techniques well-known to workers skilled in the art. For example, the ATPase activity of the co-expressed ABC transporter domains in the presence and absence of its natural “substrate” (i.e. a molecule that the protein normally transports) can be measured to determine functional activity. The ATPase activity exhibited by the co-expressed domains can then be compared to that of the native protein to determine whether the co-expressed domains have associated to form a functional transporter protein. ATPase activity assays are well-known in the art. For example, a method of measuring ATP hydrolysis using ATP labelled with a detectable label and thin layer chromatography is described by Ahn, J., and Molday, R. S. ((2000) Methods Enzymol 315, 864-79).

Detectable labels are moieties a property or characteristic of which can be detected directly or indirectly. One skilled in the art will appreciate that the detectable label is chosen such that it does not affect the ability of the protein to bind ATP. Suitable detectable labels include, but are not limited to, radioisotopes, fluorophores, chemiluminophores, colloidal particles, fluorescent microparticles and the like. Examples of labelled ATP include, but are not limited to, trinitrophenyl (TNP)-ATP (Molecular Probes, Eugene, Oreg.) and ³²P α-ATP (NEN, Boston, Mass.). One skilled in the art will understand that these labels may require additional components, such as triggering reagents, light, and the like to enable detection of the label. In one embodiment of the present invention, the substrates are labelled with a radioisotope. In another embodiment, the substrates are labelled with the radioisotope ³²P.

In accordance with the present invention, the co-expressed domains exhibit at least 40% of the ATPase activity exhibited by the native ABC transporter. In one embodiment, the co-expressed domains exhibit at least 50% of the ATPase activity exhibited by the native ABC transporter.

In addition, the co-expressed domains exhibit at least 40% of the substrate-stimulation in ATPase activity exhibited by the native ABC transporter. In one embodiment of the present invention, the co-expressed domains exhibit at least 50% of the substrate-stimulation in ATPase activity exhibited by the native ABC transporter. In other embodiments, the co-expressed domains exhibit at least 60%, at least 70% and at least 80% of the substrate-stimulation in ATPase activity exhibited by the native ABC transporter.

ATP binding can also be measured using standard techniques and compared with the ATP binding by the native transporter. For example, it has been shown that the native (or wild-type) ABCR transporter binds ATP only in the C-terminal half (see Examples). ATP binding by only the C terminal half of the co-expressed functionally complementary domains would be indicative of wild-type functionality.

The ATP binding affinity of the co-expressed functionally complementary domains can also be determined using techniques known in the art. The measured binding affinity can then be compared to that of the wild-type transporter. In general, ATP is first labelled with a detectable label. The co-expressed functionally complementary domains or the wild-type transporter is then mixed with various concentrations of the labelled substrate and the amount of bound substrate is determined. Results are analyzed by standard methods, for example through the use of Scatchard plots, and the ATP binding affinities are compared.

Methods of assaying the ability of an ABC transporter to actively transport its substrate across a membrane are also known in the art and can be employed to determine whether the co-expressed functionally complementary domains have assembled to form an active transporter. Such techniques typically use a substrate labelled with a detectable label, such as those described above.

Uses of the Method of the Present Invention

The method according to the present invention can be used to express a functional ABC transporter protein in a cell that may be defective for the protein, or to modify the existing activity of the protein in a cell. The method can find use in both research and clinical settings.

The co-expressed functionally complementary domains according to the present invention provide for a simple method for expressing a functional ABC transporter in vitro and are, therefore, useful for screening purposes, for example, for small molecule inhibitors, substrates or ligands suitable for use as therapeutics. Potential inhibitors, substrates or ligands are screened from large libraries of synthetic or natural compounds. Numerous means are currently used for random and directed synthesis of saccharide, peptide, and nucleic acid based compounds and are well-known in the art. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are also available and can be prepared according to standard procedures. Alternatively, libraries of natural compounds in the form of bacterial, fungal, plant, and animal extracts are available from, e.g., Pan Laboratories (Bothell, Wash.) or MycoSearch (North Carolina), or are readily producible. Additionally, natural and synthetically produced libraries and compounds are readily modified through conventional chemical, physical, and biochemical means.

The present invention thus provides a method of screening compounds to identify those which enhance (agonist) or block (antagonist) the action of an ABC transporter, or which bind to or act as substrates for the transporter. The method of screening may involve high-throughput techniques. For example, to screen for agonists or antagonists, a synthetic reaction mix, a cellular compartment, such as a membrane, cell envelope or cell wall, or a preparation thereof, comprising an ABC transporter formed from associated co-expressed functionally complementary domains is incubated in the presence of labelled substrate and a candidate molecule. The ability of the candidate molecule to agonize or antagonize the ABC transporter is reflected in increased or decreased binding or transport of the substrate, respectively.

Gene Therapy

The present invention also contemplates expression of the functionally complementary domains of the ABC transporter in vivo, through the use of gene therapy techniques.

As is known in the art, gene therapy includes both ex vivo and in vivo techniques. Thus host cells can be genetically engineered ex vivo with two or more nucleic acid molecules (DNA or RNA) each encoding a functionally complementary domain, with the engineered cells then being provided to a patient to be treated with the ABC transporter. In such cases, the host cells are typically autologous, so as to circumvent xenogeneic or allotypic rejection, and are usually administered to complement defects in production or activity of the ABC transporter. The cells are typically engineered with a vector comprising the nucleic acid molecule of interest. Such ex vivo methods are well-known in the art. Alternatively, cells may be engineered in vivo for expression of a polypeptide in vivo by, for example, administering one or more vectors comprising the nucleic acid molecules of interest to a patient. The nucleic acid molecules can be directly administered to a mammal by techniques known in the art, for example, as “naked” DNA (e.g. see U.S. Pat. No. 5,679,647), associated with transfection enhancing agents (e.g. see U.S. Pat. Nos. 5,739,118 and 5,837,533) or by the use of a “gene gun.” Alternatively, the nucleic acid molecules may be first incorporated into a suitable expression vector.

A number of vectors are known in the art to be suitable for gene therapy applications (see, for example, Viral Vectors: Basic Science and Gene Therapy, Eaton Publishing Co. (2000)). The vectors are typically viral-based vectors and include, but are not limited to, those derived from replication deficient retrovirus, lentivirus, adenovirus and adeno-associated virus. Retrovirus vectors and adeno-associated virus vectors are currently the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. Retroviruses, from which the retroviral vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumour virus. Specific retroviruses include pLJ, pZIP, pWE and pEM, which are well known to those skilled in the art.

The nucleic acid sequence encoding the polypeptide of the present invention is under the control of a suitable promoter. Suitable promoters which may be employed include, but are not limited to, adenoviral promoters, such as the adenoviral major late promoter, the E1A promoter, the major late promoter (MLP) and associated leader sequences or the E3 promoter; the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTR; the histone, pol III, and β-actin promoters; B19 parvovirus promoter; the SV40 promoter; and human growth hormone promoters. The promoter also may be the native promoter for the gene of interest. The selection of a suitable promoter will be dependent on the vector, the host cell and the encoded protein and is considered to be within the ordinary skills of a worker in the art.

The development of specialized cell lines (termed “packaging cells”) which produce only replication-defective retroviruses has increased the utility of retroviruses for gene therapy, and defective retroviruses are well characterised for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid molecule of the invention and renders the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable packaging virus lines for preparing both ecotropic and amphotropic retroviral systems include Crip, Cre, 2 and Am. Other examples of packaging cells include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, Vol. 1, pgs. 5-14 (1990).

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J Biol Chem 266:14143-14146). Coupling can be in the form of the chemical cross-linking with a protein or other variety (for example, lactose to convert the env protein to an asialoglycoprotein), as well as by generating fusion proteins ((for example, single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the nucleic acid molecules of the invention contained in the vector.

Another viral vector useful in gene therapy techniques is an adenovirus-derived vector. The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (for example, Ad2, Ad3, Ad7 etc.) are well known to those skilled in the art. Recombinant adenoviruses can be advantageous in certain circumstances in that they can be used to infect a wide variety of cell types, including peripheral nerve cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (for example, retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and contemplated by the present invention are deleted for all or parts of the viral E2 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127).

Compositions

The present invention also relates to pharmaceutical compositions comprising the nucleic acid molecules or expression vectors comprising the nucleic acid molecules discussed above. Thus, the nucleic acid molecules or expression vectors comprising the nucleic acid molecules of the instant invention may be employed in combination with a non-sterile or sterile carrier or carriers for use with cells, tissues, or organisms, such as a pharmaceutical carrier suitable for administration to a subject. Such compositions comprise, for instance, a media additive or a therapeutically effective amount of the nucleic acid molecules or expression vectors comprising the nucleic acid molecules of the invention and a pharmaceutically acceptable carrier or excipient. Such carriers may include, but are not limited to, saline, buffered saline, dextrose, water, glycerol, ethanol, and combinations thereof. The formulation should suit the mode of administration.

Pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philidelphia, Pa. (2000).

Administration

The nucleic acid molecules or expression vectors comprising the nucleic acid molecules of the present invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

The pharmaceutical compositions generally are administered in an amount effective for treatment or prophylaxis of a specific indication or indications. In general, the compositions are administered in an amount of at least about 10 μg/kg body weight. In most cases they will be administered in an amount not in excess of about 8 mg/kg body weight per day. Preferably, in most cases, dose is from about 10 μg/kg to about 1 mg/kg body weight, daily. It will be appreciated that optimum dosage will be determined by standard methods for each treatment modality and indication, taking into account the indication, its severity, route of administration, complicating conditions and the like.

Kits

The present invention additionally provides for kits containing the nucleic acid molecules encoding the functionally complementary domains or expression vectors comprising the nucleic acid molecules. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

For therapeutic applications, the nucleic acid molecules or expression vectors comprising the nucleic acid molecules can be in the form of pharmaceutically acceptable compositions. When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an infected area of the animal, injected into an animal, or applied to and mixed with the other components of the kit.

The components of the kit may also be provided in dried or lyophilized forms. When reagents or components are provided as a dried form, reconstitution generally is by the addition of a suitable solvent.

The invention now being generally described, it will be more readily understood by references to the following examples, which are included for purposes of illustration only and are not intended to limit the invention unless so stated.

### EXAMPLES

### Example 1

### Construction of Plasmids

cDNA fragments individually encoding the N-terminal and C-terminal halves of the human ABCR and ABCA1 protein were generated. Each half of the respective genes contains a multi-spanning membrane domain followed by a nucleotide binding domain. Each cDNA fragment was subcloned into separate pcDNA3 expression vectors (Invitrogen).

The human ABCR cDNA was generously provided by J. Nathans, Johns Hopkins University and the human ABC1 (ABCA1) was a gift of Active Pass Pharmaceuticals, Vancouver, B.C. The cDNAs were subcloned into the mammalian expression vector pcDNA3 (Invitrogen) to produce pcABCR and pcABCA1.

ABCR

The cDNAs coding for the N-half (amino acids 1-1325) and C-half (amino acids 1326-2273) of ABCR were constructed by PCR using the following primer pairs:

| | | |
|---|---|---|
| N-half- | | |
| 5′-GAGCCCTGTGGCCGGCCAGCTGTG-3′ | (FseI) and | [SEQ ID NO:1] |
| 5′-GCTCTAGATTACGGCGCCCCTGGGGAGCAGACATTGG-3′ | (XbaI); | [SEQ ID NO:21] |
| N-half-1D4- | | |
| 5′-GAGCCCTGTGGCCGGCCAGCTGTG-3′ | (FseI) and | [SEQ ID NO:3] |
| 5′-GCTCTAGATTAGGCAGGCGCCACTTGGCTGGTCTCTGTCGGCGCCCCTGGGGAGCAGACATTGG-3′ | (XbaI); | [SEQ ID NO:4] |
| C-half- | | |
| 5′-TGCTCCAAGCTTAGCATGGCTGCTCACCCAGAGGG-3′ | (HindIII) and | [SEQ ID NO:5] |
| 5′-CAGGGGTACTCCGGAAGC-3′ | (BspE1). | [SEQ ID NO:6] |

The restriction sites used to insert the PCR products are underlined with the enzyme indicated in parentheses. The bases coding for the 1D4 epitope are shown in italics. The PCR products were digested with the indicated restriction enzymes and ligated into pcABCR that had been digested with the same enzymes.

K969M and K1978M mutations were inserted by QuikChange site-directed mutagenesis (Stratagene) using PfuTurbo DNA polymerase and the following mutagenic primers (introduced mutations in bold):

| | | | |
|---|---|---|---|
| K969M- | 5′-CCACAATGGAGCTGGGATGACCACCACCTTGTCC-3′ and | | [SEQ ID NO:7] |
| | GGACAAGGTGGTGGTCATCCCAGCTCCATTGTGG-3′ | (JA13/JA14); | [SEQ ID NO:8]; |
| K1978M- | 5′-GAATGGTGCCGGCATGACAACCACATTCAAGATGC-3′ and | | [SEQ ID NO:9] |
| | 5′-GCATCTTGAATGTGGTTGTCATGCCGGCACCATTC-3′ | (JA15/JA16). | [SEQ ID NO:10] |

The AflII-ClaI (1.9 kb) and the Eco72I (0.26 kb) fragments of the resulting PCR products containing the K969M and K1978M mutations, respectively, were cloned into the original pcABCR. For the K969M/K1978M double mutant, the AflII-FseI restriction fragment of pcABCR[K1978M] was replaced with that of pcABCR[K969M].

ABCA1

The cDNA for the N-half-1D4 (amino acids 1-1302) of ABCA1 was made by replacing the 4.2 kb PmlI-XbaI fragment of pcABCA1 with a 0.9 kb PCR product amplified with the following primers:

| | | |
|---|---|---|
| 5′-CACATCTGGTTCTATGCC-3′ and | | [SEQ ID NO:11] |
| 5′-CCTCTAGATTAGGCAGGCGCCACTTGGCTGGTCTCTGTGGATTCTGGGTCTATGTC-3′ | (JA24/JA25). | [SEQ ID NO:12] |

The cDNA coding for the C-half of ABCA1 (amino acids 1303-2261) containing the 3F4 epitope was synthesized by PCR (2.9 kb) with the following primers:

| | | |
|---|---|---|
| 5′-ACTGATGCGGCCGCGGGAACATGGAATCCAGAGAGACAGACTTG-3′ and | | [SEQ ID NO:13] |
| 5′-TCCGCTAGCGTTTAAACTCATCCAGTTCGAGGGTGCAAAGGCAGATCGTATACATAGCTTTCTTTCAC-3′ | (JA29/JA30) | [SEQ ID NO:14] |
| and cloned into pCEP4 at the NotI/PmeI sites. | | |

All PCR amplified sequences were confirmed by automated DNA sequencing.

### Example 2

### Transfection of COS-1 and EBNA293 Cells

The monkey kidney fibroblast cell line COS-1 was maintained in DMEM (high glucose) supplemented with 10% fetal bovine serum. Human embryonic kidney EBNA293 cells (Invitrogen) were grown in the above DMEM containing 0.25 g/L G418. Cells were plated on 10 cm dishes and transfected the following day with 30 μg of plasmid per dish using the calcium phosphate method. The next day, cells were rinsed with 1 mM EDTA in PBS, pH 7.4, and supplied with complete medium for 24 h.

### Example 3

### Purification and Reconstitution

Membranes (from two 10 cm dishes) were solubilized in 0.5 ml of 1% Triton-X100 in Buffer A (140 mM NaCl, 20 mM Tris-HCl, pH 7.4) for 20 min on ice. For ATPase assays, the membrane preparation step was omitted and the cell suspension was solubilized directly in Buffer B (10 mg/ml soybean phospholipids, 10% glycerol, 1 mM dithiothreitol, 100 mM NaCl, 3 mM MgCl₂, 50 mM NaHEPES, pH 7.4) containing 18 mM CHAPS. The supernatant after a 10 min centrifugation at 40,000 rpm (TLA100.4 rotor) was mixed with 50 μl Rim3F4 Sepharose 2B for 1 h at 4° C. The beads were washed 6 times in Buffer A containing 0.2% Triton X-100 or Buffer B containing 10 mM CHAPS and eluted with 4% SDS (for electrophoresis) or 0.2 mg/ml Rim3F4 peptide (for reconstitution and determination of ATPase activity). Purified protein (24 μl) was incubated with 6 μl of 50 mg/ml lipid (1:1 mixture of dioleoylphosphatidylethanolamine and brain polar lipid, by weight) and 3 μl n-octylglucoside for 30 min on ice. The mixture was diluted rapidly with 200 μl of Buffer C (1 mM dithiothreitol, 140 mM NaCl, 25 mM NaHEPES, pH 7.4) and passed through a 200 μl Extracti-gel column (Pierce). The flow-through containing the reconstituted protein was used for determination of ATPase activity.

### Example 4

### Localization of ABCR in Transfected COS-1 Cells by Indirect Immunofluorescence Microscopy

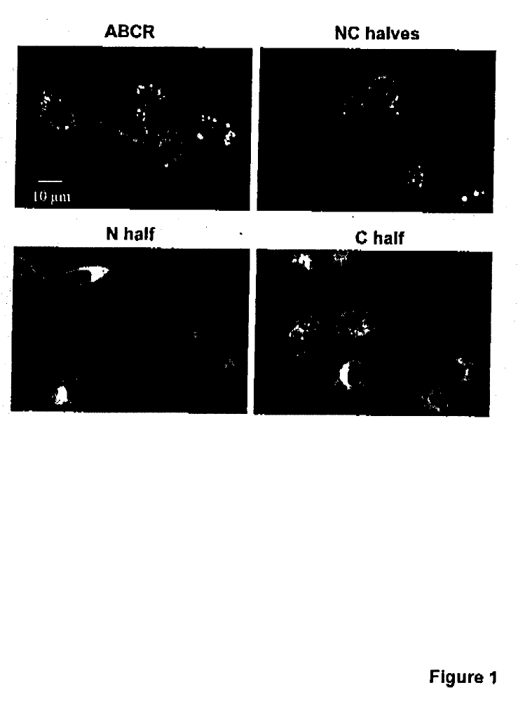

The subcellular distribution of full length ABCR and the N and C half-molecules expressed in COS-1 cells was determined by immunofluorescence microscopy (FIG. 1). Rather than localizing predominantly in the endoplasmic reticululm (ER) and Golgi, which is expected for transiently overexpressed intracellular membrane proteins, ABCR was typically associated with intracellular vesicles of varying sizes. Clusters of 2-4 large vesicles were observed in some cells, while numerous small vesicles spread throughout the cytoplasm were seen in other cells. These intensely labeled vesicles do not appear to be artifacts since mutating a single amino acid in ABCR (D846H) changed the distribution from vesicular to perinuclear reticular distribution characteristic of misfolded proteins retained in the ER. ABCR did not co-localize with a number of organelle markers (catalase for peroxisomes, LAMP-2 for late endosomes, LysoTracker for lysosomes). The expression pattern of the N-half or C-half when expressed alone was mostly perinuclear indicative of ER localization. However, when the two halves were co-expressed, a significant fraction of the protein was found in vesicular structures like those seen in cells transfected with wild-type, full-length ABCR.

### Example 5

### Functional Protein Activity

1) Association of the Two Halves of ABCR when Co-Expressed

Non-reduced samples were prepared by solubilizing cells in the presence of 100 mM n-ethylmaleimide to prevent formation of secondary disulfide bonds. Proteins were separated on 6% polyacrylamide gels, stained with Coomassie brilliant blue, destained in 10% acetic acid and soaked in water. The gel was dried under vacuum and exposed to a storage phosphor screen or autoradiography film. For Western blot analysis, the electrophoresed proteins were transferred to an Immobilon-P membrane which was subsequently blocked in 1% nonfat milk and incubated with primary and peroxidase-conjugated secondary antibodies. Duplicate samples were loaded on the same gel and analyzed on Western blots using Rim3F4 (1:10 dilution) and Rim5B4 (1:100 dilution) antibodies.

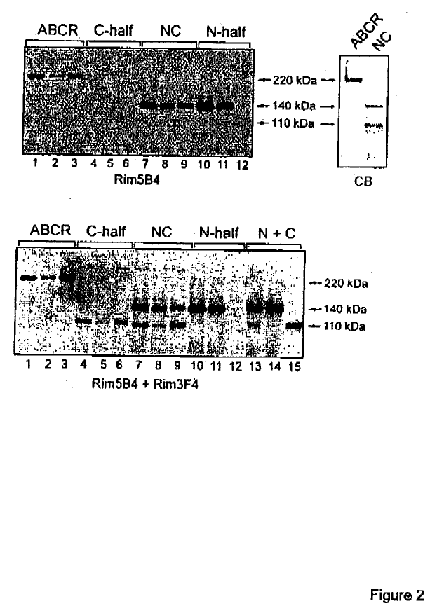

The N and C halves of ABCR, each containing a transmembrane domain followed by an NBD, were expressed individually by single transfections or together by co-transfection in COS-1 cells. FIG. 2 shows Western blots of COS-1 cell extracts, flow-through (unbound) fractions, and peptide-eluted (bound) fractions of the expressed full-length ABCR (˜220 kDa) and the N (˜140 kDa) and C (110 kDa) half-molecules isolated on a Rim3F4-Sepharose matrix specific for an epitope near the C-terminus of ABCR(2). When the two halves were co-expressed (NC), about 50% of the N-half (detected with the Rim5B4 MAb) co-purified with the C-half (detected with the Rim3F4 MAb), while the remainder was in the flow-through fraction. The N-half by itself did not bind to the Rim3F4-Sepharose matrix nor did it co-purify with the C-half when the N and C halves were individually expressed and mixed together prior to immunoaffinity purification. Coomassie blue stained gels showed that full-length ABCR and co-expressed/co-purified N and C halves were the predominant proteins observed in the peptide-eluted fraction from the Rim 3F4 immunoaffinity column.

2) ATPase Activity

ATPase activity was measured as described previously (Ahn, J., Wong, J. T., and Molday, R. S. (2000) J Biol Chem 275(27), 20399-405) using 50 μM [α³²P]ATP and thin layer chromatography. The all-trans retinal concentration was determined spectrophotometrically (λ_{383 nm}=42.88 mM⁻¹ cm⁻¹). Protein concentration was estimated from the eluate before reconstitution by laser densitometry of Coomassie blue stained gels using bovine serum albumin as a standard. This method gives an overestimation of the actual protein content after reconstitution (hence lower specific activity) since recovery from the Extracti-gel column is less than 100 percent. Direct protein measurements after reconstitution by densitometry of Western blots was about half of that in the eluate. However, the latter method gave variable results, so protein concentration after reconstitution was extrapolated from that in the eluate assuming 100% recovery.

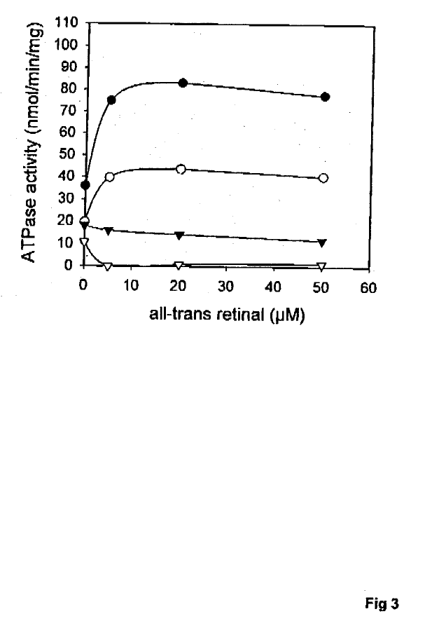

ATPase Activity of Expressed N and C Halves—The basal and retinal activated ATPase activity of full-length ABCR and the N and C halves individually or co-expressed in COS-1 cells was determined after immunoaffinity purification and reconstitution into lipid vesicles. FIG. 3 shows that both the full-length ABCR and co-expressed N and C halves purified on a Rim 3F4 column were stimulated 1-2 fold by all-trans retinal. The specific activity of the full-length protein, however, was generally higher than the co-expressed half molecules. In contrast, the ATPase activity of the individually expressed C half and N half containing a nine amino acid 1D4 epitope tag (N*) required for immunoaffinity purification was not stimulated by retinal. The 1D4 tag did not affect the functional interaction of the N and C halves, since co-expression of the N* and C halves gave similar basal and retinal stimulated activity as co-expression of the untagged N and C halves (data not shown).

3) Preferential Azido-ATP Labelling

Preparation of Membranes—Membranes from transfected cells were prepared as described previously (Bungert, S., Molday, L. L., and Molday, R. S. (2001) J Biol Chem 276(26), 23539-46). In some experiments, the protocol was scaled down and the cell homogenate from one or two 10-cm dishes, after passing through a 26-gauge needle (6 times), was centrifuged on a discontinuous gradient consisting of 5% and 60% sucrose for 30 min at 30,000 rpm (60,000×g) in a TLS55 rotor (Beckman Optima TL ultracentrifuge).

8-Azido-ATP Photoaffinity Labeling—Membranes (50-150 μg protein) in 50 μl of labeling buffer (20 mM Tris-HCl, pH 7.4, 140 mM NaCl, 5 mM MgCl₂) were incubated with 1-4 μM 8-azido-[α-³²P]ATP (Perkin-Elmer Life Science; 20 Ci/mmol) on ice, with gentle shaking, under UV light (254 nm) for 10 min at a distance of 10 cm. Ice-cold 20 mM Tris-HCl, pH 7.4, was added and the membranes were collected by centrifugation (TLA45 rotor, 55000 g, 15 min). The membrane suspension (200 μl in TBS [Tris-HCl, pH 7.4, 140 mM NaCl]) was added to 200 μl of 2% Triton X-100 in TBS, pH 7.4). After 30 min on ice, the cleared extract was mixed with 25 μl antibody coupled to Sepharose 2B for 1-12 h at 4° C. The beads were washed 4 times in TBS containing 0.2% Triton X-100 and eluted twice, 30 μl each, in 4% SDS, 0.2% Triton X-100, TBS.

Trypsin Cleavage of Bovine ABCR—Bovine ROS were isolated as previously described (Molday, R. S., and Molday, L. L. (1987) J Cell Biol 105(6 Pt 1), 2589-601) and treated with 1.6-4.0 μg/ml trypsin for 30 min at 0° C. (Bungert, S., Molday, L. L., and Molday, R. S. (2001) J Biol Chem 276(26), 23539-46). The reaction was stopped by the addition of 5-fold excess of soybean trypsin inhibitor.

8-Azido-ADP Photoaffinity Labeling—Thoroughly washed ROS membranes were labeled with 5 μM 8-azido[α-³²P]ADP (Affinity Labeling Technologies, 16.8 Ci/mmol) for 15 to 30 min as described for 8-azido-ATP binding. DTT at a final concentration of 10 mM was added to quench the reaction. After 15 min, ice-cold Tris-EDTA buffer (0.5 mM EDTA, 10 mM Tris-HCl, pH 7.4) was added and the membranes were washed 5 times by centrifugation at 30,000 rpm for 15 min (TLA45 rotor in a Beckman Optima TL-100 centrifuge). The membranes were suspended in 50 μl of Tris-EDTA buffer and an equal volume of buffer containing trypsin (4 μg) was added. After 30 min on ice, the membranes were treated with trypsin inhibitor (50 μg). Cold Tris-EDTA, pH 7, buffer was added and the membranes pelleted by centrifugation (30000 rpm, 15 min in a TLA 45 rotor). The membranes were resuspended in 50 μl of Tris-EDTA buffer, and an equal volume of SDS sample buffer with β-mercaptoethanol was added. Thirty μl samples were loaded in triplicate onto three 8% SDS gels: two gels were used for Western blot analysis with Rim3F4 and Rim5B4 antibodies, respectively, and the third gel was stained, destained, dried and analyzed for ³²P labeling with a PhosphorImager.

To remove tightly bound nucleotides, membranes were treated as follows (Hyndman, D. J., Milgrom, Y. M., Bramhall, E. A., and Cross, R. L. (1994) J Biol Chem 269(46), 28871-7). The washed membrane pellet (4 mg) was resuspended in 1 ml of 100 mM Na₂SO₄, 50% glycerol, 3 mM MgCl₂, 50 mM NaHEPES, pH 7.5, and dialyzed against 3 changes of the same buffer at 4° C. (2×500 ml for 3 h each, 1000 ml overnight). The sample (100 μl) was diluted with labeling buffer (20 mM Tris-HCl, pH 7.4, 140 mM NaCl, 5 mM MgCl₂). The washed pellet was resuspended and photolabeled as described above.

The washed membranes (4 mg) were resuspended in 1 ml of 50 mM NaHEPES, pH 7.5, 100 mM Na₂SO₄, 50% glycerol, 3 mM MgCl₂ and 10 mM CHAPS, stirred in a glass tube at 4° C. for 1 h, and dialysed against 3 changes of resuspension buffer. The solubilized membranes, 100 μl, were centrifuged through a column of Sephadex G-50 (previously equilibrated with labeling buffer). The volume was made up to 100 μl with labeling buffer and the sample was photoaffinity labeled as described above. This was then passed through another column centrifugation procedure to remove unbound label. Half of the labeled sample was subjected to trypsin digestion as described above.

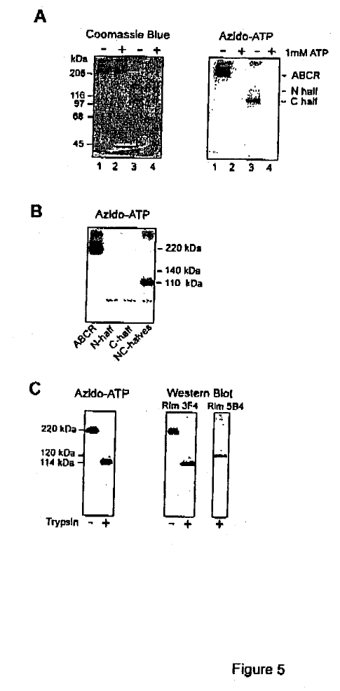

To determine whether ATP binds to one or both NBDs of ABCR, membranes from transfected cells were photoaffinity labeled with 8-azido-[³²P]ATP, and expressed ABCR proteins were purified by immunoaffinity chromatography. FIG. 5 shows that when the N and C halves were expressed together, only the C-half was labeled with 8-azido-ATP. This labeling was essentially abolished by the addition of 1 mM ATP prior to photoaffinity labeling (FIG. 5A, lanes 2 and 4). When expressed individually, the N-half bound ATP weakly and the C-half did not bind ATP at all (FIG. 5B).

To determine if the C-half of native ABCR also selectively bound ATP, ABCR in ROS disk membranes was cleaved with trypsin to generate N and C half molecules of similar size (Illing, M., Molday, L. L., and Molday, R. S. (1997) J Biol Chem 272(15), 10303-10; Bungert, S., Molday, L. L., and Molday, R. S. (2001) J Biol Chem 276(26), 23539-46) for photoaffinity labeling with 8-azido-[³²P]ATP. Only the C-half (˜114 kDa) was labeled (FIG. 5C) as found for the co-expressed N and C half molecules of ABCR. Identical results were obtained when full-length bovine ABCR was photoaffinity labeled with 8-azido [³²P]ATP prior to trypsin cleavage (data not shown). This confirms that both the co-expressed N and C half molecules of ABCR and the native protein are functioning in the same way.

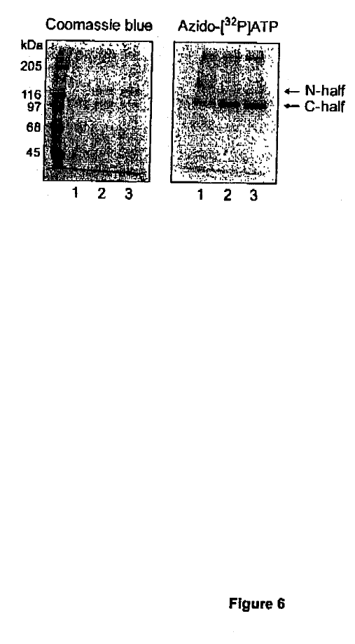

The possibility that more C-half than N-half is recovered on Rim3F4 beads and therefore displays more azido-ATP label was investigated. This is unlikely to be a problem since it has already been shown that any excess C-half which is not associated with N-half does not label with azido-ATP (FIG. 5B). Nevertheless, FIG. 6 lane 3 shows that when the two halves of ABCR were co-immunoprecipitated with Rim5B4 (which binds the N-half of ABCR and should only purify C-half that is bound to the N-half), the C-half is still labeled more strongly.

4) Azido-ATP/ADP Trapping by Co-Expressed N and C Halves of ABCR

8-Azido-ATP Trapping—Membranes were incubated with 5 [M 8-azido-[α-³²P]ATP in 50 μl of labeling buffer with or without 800 μM sodium orthovanadate for 10 min at 37° C. All-trans retinal and 50 μM DTT were added where indicated. Binding was stopped by the addition of ice-cold 20 mM Tris-HCl, pH 7.4, the membranes were collected by centrifugation and washed one more time. Samples exposed to orthovanadate were washed in the presence of 800 μM orthovanadate. In some experiments MgATP (10 mM) was included in the wash step, but had no effect. The membranes were suspended in 30 μl of 20 mM Tris-HCl, pH 7.4, irradiated with UV light for 10 min on ice, diluted to 200 μl with TBS, pH 7.4, and solubilized as described above for azido-ATP binding.

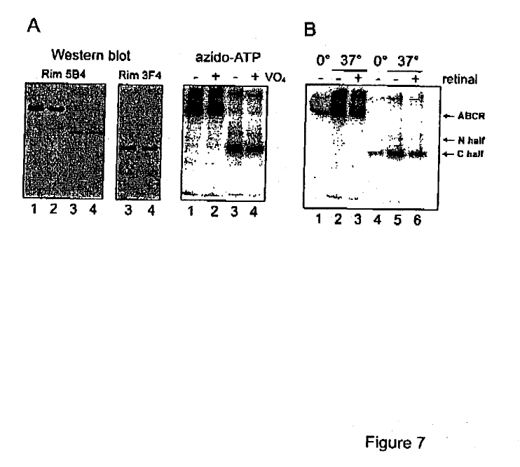

To gain more insight into the properties of the NBD of ABCR, trapping experiments were carried out using 8-azido-[α³²P]ATP. As shown in FIG. 7, more 8-azido ATP/ADP was trapped by ABCR under hydrolyzing conditions (37° C.) than at 0° C. This binding was not dependent on sodium orthovanadate or influenced by the presence of all-trans retinal (FIG. 7). Co-expression of the N and C halves further revealed that as with ATP binding, ATP/ADP trapping occurred in NBD2 of the C-half, confirming that the co-expressed protein retained the functionality of the native ABCR protein.

### Example 6

### 8-Azido-ATP Binding by Co-Expressed N and C Halves of ABCA1

It has been established that the N-terminal NBD (NBD1) of MRP1, CFTR, SUR1 or the NBD of TAP1 is responsible for high affinity ATP binding and the C-terminal NBD (NBD2) or TAP2 is more important for ATP hydrolysis and ADP trapping. The unexpected finding that ATP binding occurs only on the C half of ABCR, prompted the examination of the ATP binding properties of ABCA1, a member of the ABCA subfamily which is most similar to ABCR.

Membranes from cells expressing the N and C halves of ABCA1 engineered to contain the 3F4 epitope in the C-half and ABCR were photoaffinity labeled with azido-[α-³²P]ATP, isolated by immunoprecipitation using Rim3F4-Sepharose 2B beads and analyzed on an SDS gel.

FIG. 6 shows that the two halves of ABCA1 were labeled equally well in contrast to ABCR. FIG. 6 also demonstrates that isolation of the co-expressed halves of ABAC1 using Rim3F4, which only binds the C-terminal half of the protein, also isolated the N half of the protein indicating that the two halves of ABCA1 associate when co-expressed.

### Example 7

### Effect of Walker A Lysine-to-Methionine Mutations on ATP Hydrolysis and Binding

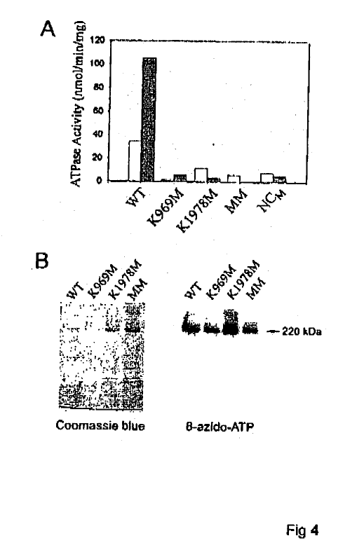

The conserved lysine residue in the NBD Walker A motif of ABC proteins is critical for the hydrolysis of ATP. Mutation of this lysine to methionine in P-glycoprotein abolishes basal and drug-stimulated ATPase activity (35,36). With ABCR, the lysine-to-methionine substitution in the NBD1 (K939M) and NBD2 (K1978M) or in both (K939M/K1978M) significantly reduced the basal ATPase activity of ABCR, and abolished retinal activation (FIG. 4A). In an analogous manner, retinal-stimulated ATPase activity was also abolished when the N-half was co-expressed with the K1978M C-half mutant (amino acid numbers represents that of the full-length ABCR), again showing that the functionality of the co-expressed transporter and the native protein are the same.

The ATP binding of these mutants was also examined using the photoreactive ATP analogue 8-azido-α-³²P]ATP. The photoaffinity labeling intensities of the single mutants (K969M and K1978M) were similar to wild-type ABCR relative to the amount of purified ABCR stained with Coomassie blue (FIG. 4B). A small reduction in labeling, however, was observed for the K969M/K1978M double mutant.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A nucleic acid composition for expression of a functional ABCR transporter in a host cell, said nucleic acid composition comprising two or more different nucleic acid molecules, each nucleic acid molecule encoding one or more domains of an ABCR transporter, wherein said at least one of the domains encoded by each nucleic acid molecule are functionally complementary.

2. The nucleic acid composition according to claim 1, wherein said two or more nucleic acid molecules are associated with a lipid.

3. The nucleic acid composition according to claim 1, wherein said two or more nucleic acid molecules are provided in separate expression vectors.

4. The nucleic acid composition according to claim 1, wherein said two or more nucleic acids are operatively associated with one or more regulatory elements.

5. A host cell comprising the nucleic acid composition according to claim 1.

6. A method of expressing a functional ABCR transporter in a host cell comprising transforming or transfecting said host cell with the nucleic acid composition according to claim 1.

7. A system for expressing an ABCR transporter in a host cell comprising two or more expression vectors, each expression vector comprising a different nucleic acid molecule and each nucleic acid molecule encoding one or more domains of an ABCR transporter, wherein said at least one of the domains encoded by each nucleic acid molecule is a functionally complementary domain, and
wherein, upon co-expression in said host cell, the functionally complementary domains associate to provide a functional ABCR transporter.

8. The system according to claim 7, wherein said two or more expression vectors further comprise one or more regulatory elements operatively associated with said nucleic acid molecule.

9. The system according to claim 7, wherein said two or more expression vectors are plasmids.

10. The system according to claim 7, wherein said two or more expression vectors are viral vectors.

11. The system according to claim 7, wherein said one or more domains comprise a nucleotide binding domain (NBD).

12. The system according to claim 11 wherein said one or more domains further comprise a multi-spanning membrane domain (MSD).

13. The system according to claim 7, wherein said one or more domains comprise a multi-spanning membrane domain (MSD).

14. The system according to claim 7, wherein at least one of said domains comprise an epitope.

15. The system of claim 14, wherein said epitope is selected from the group consisting of: 3F4, 5B4, and 1D4.

16. A host cell comprising the system according to claim 7.

17. A method for expressing an ABCR transporter in a host cell comprising:
(a) transforming or transfecting said host cell with two or more expression vectors, each expression vector comprising a different nucleic acid molecule and each nucleic acid molecule encoding one or more domains of an ABCR transporter; and
(b) culturing said host cell under conditions that allow for expression of said one or more domains.

18. The method according to claim 17, wherein said host cell is a prokaryotic cell.

19. The method according to claim 17, wherein said host cell is a eukaryotic cell.

20. The method according to claim 17, wherein said functional ABCR transporter forms at least 20% of the total recombinant protein produced by said cell.

21. The method according to claim 17, wherein said functional ABCR transporter exhibits at least 50% of the ATPase activity of the native ABCR transporter.

22. A kit for expressing an ABCR transporter in a host cell comprising:
(a) the nucleic acid composition according to claim 1 or the system according to claim 7;
(b) one or more containers, and optionally
(c) instructions for use.


SEQUENCE LISTING (14 sequences)

SEQ ID NO: 1 - DNA, 24 bp, Homo sapiens
gagccctgtggccggccagctgtg

SEQ ID NO: 2 - DNA, 37 bp, Homo sapiens
gctctagattacggcgcccctggggagcagacattgg

SEQ ID NO: 3 - DNA, 24 bp, Homo sapiens
gagccctgtggccggccagctgtg

SEQ ID NO: 4 - DNA, 64 bp, Homo sapiens
gctctagattaggcaggcgccacttggctggtctctgtcggcgcccctggggagcagacattgg

SEQ ID NO: 5 - DNA, 35 bp, Homo sapiens
tgctccaagcttagcatggctgctcacccagaggg

SEQ ID NO: 6 - DNA, 18 bp, Homo sapiens
caggggtactccggaagc

SEQ ID NO: 7 - DNA, 34 bp, Homo sapiens
ccacaatggagctgggatgaccaccaccttgtcc

SEQ ID NO: 8 - DNA, 34 bp, Homo sapiens
ggacaaggtggtggtcatcccagctccattgtgg

SEQ ID NO: 9 - DNA, 35 bp, Homo sapiens
gaatggtgccggcatgacaaccacattcaagatgc

SEQ ID NO: 10 - DNA, 35 bp, Homo sapiens
gcatcttgaatgtggttgtcatgccggcaccattc

SEQ ID NO: 11 - DNA, 18 bp, Homo sapiens
cacatctggttctatgcc

SEQ ID NO: 12 - DNA, 56 bp, Homo sapiens
cctctagattaggcaggcgccacttggctggtctctgtggattctgggtctatgtc

SEQ ID NO: 13 - DNA, 44 bp, Homo sapiens
actgatgcggccgcgggaacatggaatccagagagacagacttg

SEQ ID NO: 14 - DNA, 68 bp, Homo sapiens
tccgctagcgtttaaactcatccagttcgagggtgcaaaggcagatcgtatacatagctttctttcac